(19) United States
(12) Patent Application Publication
(10) Pub. No.: US 2014/0018637 A1
(43) Pub. Date: Jan. 16, 2014

(54) Cloud-Based Monitoring of Medical Devices

(71) Applicant: Oakwell - Cayman Company, Grand Cayman (KY)

(72) Inventors: John A. Bennett, Villanova, PA (US); Ian Geiser, West Chester, PA (US); John-Yuhan Bai, San Gabriel, CA (US)

(21) Appl. No.: 13/924,106

(22) Filed: Jun. 21, 2013

Related U.S. Application Data
(63) Continuation-in-part of application No. 13/831,480, filed on Mar. 14, 2013.
Application No. 13/831,480 is a continuation-in-part of application No. 13/547,502, filed on Jul. 12, 2012.

(51) Int. Cl. A61B 5/00; A61H 9/00; A61M 1/00

(52) U.S. Cl. CPC A61B 5/0022; A61M 1/0088; A61H 9/0078
USPC 600/301; 607/5; 607/51; 604/319; 601/152; 601/15

(57) ABSTRACT

A system includes a communications device integrally incorporated into a remotely deployed medical device to monitor the operative status and use of the device. A home-based medical device will communicate directly through an integrated communications device to provide informational data to a Cloud server or professional caregiver without requiring input or activity by the patient. A remotely deployed medical device, such as a defibrillator, will provide periodic reports regarding the operational status, including remaining battery life, of the deployed device, thus allowing a service technician to provide maintenance service only when service is needed. Although the informational data is preferably send to a Cloud computing and storage site, the communications device can be accessed remotely and directly queried to provide requested informational data thus providing real time access to the status and utilization of the medical device and allowing the professional caregiver access to change operational parameters for the medical device.

### CROSS-REFERENCE TO RELATED APPLICATIONS

This application is a continuation-in-part of U.S. patent application Ser. No. 13/831,480, filed on Mar. 14, 2013, which is a continuation-in-part of U.S. patent application Ser. No. 13/547,502, filed on Jul. 12, 2012, which claims domestic priority from U.S. Provisional Application Ser. No. 61/511,080, filed on Jul. 24, 2011, the contents of which are incorporated herein by reference.

### FIELD OF THE INVENTION

The present invention relates generally to an apparatus and an associated system for monitoring of and analyzing the data resulting from the utilization or deployment of an operable device, particularly medical devices that are used to ascertain information related to the health of a person and, more specifically, medical devices used in therapy outside of a hospital or office of a professional health caregiver.

### BACKGROUND OF THE INVENTION

Medical devices can be utilized to ascertain a specific health related condition of a patient. Such devices include, among others, devices for determining a person's blood pressure, a thermometer to measure a person's temperature, an oximeter that identifies the oxygen level in a patient's blood, and a glucose meter for identifying the level of a person's blood sugar. Other medical devices are deployed for emergency utilization, such as portable defibrillators that can be utilized to provide an electrical current to restore a person's heart rhythm. Still other medical devices are used in home therapy, such as a negative pressure wound therapy systems, or inflatable garment therapy systems, both of which can be coupled to a pump to provide specific therapy for the patient without requiring the patient to be hospitalized, or even to have a professional health caregiver be present during the application of the therapy.

When a patient is hospitalized, the patient is typically connected to several medical devices that often automatically test a specific physical condition of the patient while the patient remains in the hospital bed. Such medical devices would include a blood pressure cuff that periodically inflates to measure the patient's blood pressure; an oximeter that can continuously monitor the oxygen level of the patient's blood, which provides an indication of how effective the patient's lungs are working to infuse oxygen into the patient's blood; and a thermometer that periodically or continuously records the patient's temperature to provide an indication of infection. Periodically, a hospital nurse visits the patient and records the numbers associated with the operation of these medical devices and transfers those numbers to the patient's chart.

Publically deployed medical devices, specifically defibrillators, provide access to potentially life-saving medical therapy even when the person suffers from an emergency medical condition in a public place, such as a shopping mall or an airport terminal. As is noted in an article in the Wall Street Journal entitled “F.D.A Seeks to Toughen Defibrillator Regulations”, dated Mar. 22, 2013, publically deployed defibrillators have experienced a number of failures for reasons that include improper maintenance, including software failures and battery failures. Furthermore, defibrillators can be used to ascertain the heart rate, heart rhythm, etc. of the person before the defibrillator is utilized. If the person using the defibrillator understands the information derived from the sensors, that person could be able to determine whether the defibrillator should be used or not and, perhaps, in what capacity.

Medical devices for applying home-based therapy are typically used on the honor system by the patient. The health care professional does not know if the medical therapy device is being used at all, or whether the medical device is being used in the proper manner. Furthermore, the health care provider rarely knows how effective the home-based medical therapy device is performing for the patient. Accordingly, a visiting nurse will stop at the patient's home to check on the patient periodically, perhaps once every week or two, and the health care provider will likely see the patient less frequently than the visiting nurse. However, this therapy should be conducted multiple times each day. Unfortunately, the health care provider typically has little or no feedback as to whether the therapy is actually being done, or if the therapy is producing positive results. Further, the patient has no feedback as to the effectiveness of the therapy. Particularly since positive feedback will normally stimulate and encourage the continued use of the therapy, providing the patient with ongoing results of the therapy has a synergistic effect on the effectiveness of the therapy.

In U.S. Pat. No. 7,231,263, granted to Soo Bong Choi on Jun. 12, 2007, a control system for utilizing an insulin pump is disclosed. The Choi system operates to provide security and control over the operation of an insulin pump. Before the pump can be utilized, the person logging in to the system provides an appropriate identification. The blood sugar level for the patient is ascertained and the amount of insulin to be injected is adjusted when the person logged in is the patient's health care provider. A Bluetooth module is utilized to make the communication between the insulin pump module and the internet.

A remote control for a medical apparatus is disclosed in U.S. Pat. No. 6,768,425, issued on Jul. 27, 2004, to J. Christopher Flaherty, et al, wherein the medical treatment apparatus has a local processor having a communications unit associated with it. The hand remote control includes a remote processor and an associated remote communication component that can communicate with the local processor in a wireless manner. Accordingly, the medical device can be operated with the operation of the remote control device without requiring the patient to manually access the medial apparatus.

In U.S. Patent Application Publication No. 2007/0249976, published on Oct. 25, 2007, a control apparatus for an inflatable garment therapy device is disclosed wherein the pump apparatus is self-contained within therapy device. U.S. Patent Application Publication No. 2007/0049853, published on Mar. 1, 2007, discloses the inflatable garment therapy device used to improve blood circulation in patient's extremities. Similarly, U.S. Pat. No. 6,852,089, issued on Feb. 8, 2005, to Richard J. Kloecker, et al, discloses an inflatable garment therapy device for a patient's arms. In U.S. Pat. No. 6,736,786, granted to Paul Shabty on May 18, 2004, a counter-pulsation device is disclosed in which the operation of the inflatable cuff is coupled to a microprocessor to take the EKG of the patient so that the cuff inflation can be timed with the patient's EKG.

It is known that certain medical devices can be connected to a smart phone via a WiFi connection or through the operation of an app that allows the smart phone to communicate with a health care professional who has asked for the information to be forwarded. However, the collection of data is sporadic and requires the patient to manipulate the medical device and/or the smart phone to be able to transmit the pertinent data from the medical device to the health care professional. One such device is a glucose monitor provided by Johnson & Johnson, which connects via an app on a smart phone to transmit the blood sugar level directly to the health care professional.

It would be desirable to provide a system for monitoring the operation and/or capabilities of a medical device without requiring the cooperative input from a person, specifically from the person using the medical device, so that any data transmitted from the medical device is transmitted as desired. It is also desirable that the data collected from the medical device is sent to a computer, such as a Cloud server, where analysis of the data can be performed and a report sent to the health care professional in a predetermined manner. It is also desirable that the medical device have incorporated therein the apparatus by which communication of data related to the operation and/or capabilities of the medical device is transmitted automatically, or when remotely queried from the health care professional.

### SUMMARY OF THE INVENTION

It is an object of this invention to overcome the disadvantages of the prior art by providing a system for monitoring the use of medical devices.

It is another object of this invention to provide a monitoring system that can track and record the utilization of therapy incorporating medical devices, such as an inflation garment for intermittent pneumatic compression therapy or negative pressure wound therapy.

It is a feature of this invention that the utilization information can be transmitted to a remote server for access by medical professionals.

It is another feature of this invention that the medical device can be programmed to provide a notification to predetermined medical professionals when certain utilization parameters are encountered.

It is another advantage of this invention that the medical apparatus can report to a central station monitored by caregivers as to the timing and duration of use of the medical device by the patient.

It is still another feature of this invention that the medical apparatus can report a malfunction of the medical apparatus to a central station.

It is still another advantage of this invention that the caregiver monitoring the central station can be alerted to any malfunction in the operation of the medical apparatus so that repair or replacement of the medical apparatus can be arranged promptly.

As a caregiver or healthcare professional, one can access the data cloud through multiple modalities, such as a smart phone application, a computer program, a website, or an online portal of a hospital intranet.

It is yet another feature of this invention that the effectiveness of the therapy utilizing the medical device can be monitored by professional caregivers.

It is still another feature of this invention that the professional medical caregiver can query the medical device directly to obtain a status report therefrom.

It is yet another advantage of this invention that changes in the operative parameters of the medical device can be determined from a remote location through monitoring of the central station to which information about the operation of the medical apparatus is transmitted.

It is still another object of this invention to provide a monitoring of the use and operation of a medical apparatus used in patient therapy in a home environment.

It is yet another object of this invention to place an RFD tag on the medical apparatus so that the medical apparatus can be located and identified.

It is a further feature of this invention that the medical apparatus can be provided with a selected communication technology to report the use and operation of the medical apparatus to a central station or directly to a professional caregiver.

It is yet another advantage of this invention that the professional caregiver could also be passively notified by the data center of any of the informational data through a text message, a phone call, a pager message, a push mail on a smart phone, or a pop up notification on a computer screen or smart phone screen.

It is still another advantage of this invention that the professional caregiver can provide intervention to the patient through various models through the cloud computing pathway, including the sending of a command back to the medical device using a smart phone or a computer to remotely control the medical device to start or stop, or to change settings, or to directly intervene with the patient by giving the patient a phone call upon the receipt of an error message by caregiver, or performing an onsite visit if situation warrants.

It is a further advantage of this invention that the communication technology can be used to communicate by Bluetooth® technology through a telephone system, to a smart phone or via the Internet.

It is yet another feature of this invention that the medical device has incorporated therein a communications device that communicates directly with the recipient, including the professional medical caregiver and/or a remote Cloud server.

It is another advantage of this invention that input or activity from the patient using the medical device is not required for the medical device to report information directly to the remote Cloud server for storage thereon.

It is still another advantage of this invention that the collection of medical information stored on a Cloud server is coded in a manner that only the appropriate professional caregiver can associate with any specific patient.

It is yet another advantage of this invention that the coded information stored on the Cloud server can be utilized for medical research purposes without exposing the identity of the patients.

It is still a further advantage of this invention that the data reported from the medical device can be associated with predictive modeling software to assist the professional caregiver with analysis and evaluation of the patient's condition and prognosis.

It is yet a further advantage of this invention to utilize a secure packet to transmit information relating to the use and effectiveness of medical apparatus via a cellular network directly to a Cloud computing and storage site.

It is another advantage of this invention that medical professionals and caregivers can more effectively treat the patients during home visits because of the advance knowledge from a central station related to the use, operation and effectiveness of the medical therapy utilizing a monitored medical apparatus.

It is still another feature of this invention that the Cloud computing and storage site stores data related to the utilization and effectiveness of the medical apparatus being monitored so that the medical professional can access that stored information as needed to effectively monitor the patient's therapy.

It is still another advantage of this invention that the Cloud computing and storage site can provide an alert to the pertinent medical professional if the transmitted data is not within predetermined limits.

It is yet another object of this invention to monitor the operation of a medical devices to detect the operation of the device, the length of time the device is operated, the effectiveness of the device and any malfunctions in the operation of the device.

It is an advantage of this invention that the informational data received from a medical device could include ON/OFF flags, the duration where the therapy was on, intentional interruption of therapy, erratic therapy behavior, or error messages, and for a negative wound therapy device or an intermittent pneumatic compression therapy device, the informational data received can include ON/OFF flags for the device and prolonged irregular inactivity of the device, which informational data will be transmitted to the data cloud.

It is a feature of this invention that a communication device can be incorporated into defibrillators to permit informational data to be reported directly to a central server or to be remotely queried to determine the status of the defibrillator, including the operative ability of the defibrillator and the remaining life of the battery.

It is an advantage of this invention that a service technician need only to visit a deployed defibrillator when the communications device signals that service or maintenance is needed for the defibrillator.

These and other objects, features and advantages are accomplished according to the instant invention by providing a system that includes a communications device integrally incorporated into a remotely deployed medical device to monitor the operative status and use of the device. A home-based medical device will communicate directly through an integrated communications device to provide informational data to a Cloud server or professional caregiver without requiring input or activity by the patient. A remotely deployed medical device, such as a defibrillator, will provide periodic reports regarding the operational status, including remaining battery life, of the deployed device, thus allowing a service technician to provide maintenance service only when service is needed. Although the informational data is preferably send to a Cloud computing and storage site, the communications device can be accessed remotely and directly queried to provide requested informational data thus providing real time access to the status and utilization of the medical device and allowing the professional caregiver access to change operational parameters for the medical device.

### BRIEF DESCRIPTION OF THE DRAWINGS

The foregoing and other objects, features, and advantages of the invention will appear more fully hereinafter from a consideration of the detailed description that follows, in conjunction with the accompanying sheets of drawings. It is to be expressly understood, however, that the drawings are for illustrative purposes and are not to be construed as defining the limits of the invention.

FIG. 1 is a schematic perspective view of an inflation garment therapy apparatus incorporating the principles of the instant invention;

FIG. 2 is a schematic diagram of the system incorporating the principles of the instant invention;

FIG. 3 is a schematic perspective view of a contoured negative pressure bandage for toes as depicted in FIG. 1;

FIG. 4 is a schematic representation of a cervical electromagnetic bone growth stimulation apparatus incorporating the principles of the instant invention;

FIG. 5 is a schematic representation of a non-cervical electromagnetic bone growth stimulation apparatus; and

FIG. 6 is a schematic representation of a defibrillator incorporating a communications device to directly connect the defibrillator to a remote server to report operational status of the defibrillator.

### DETAILED DESCRIPTION OF THE PREFERRED EMBODIMENT

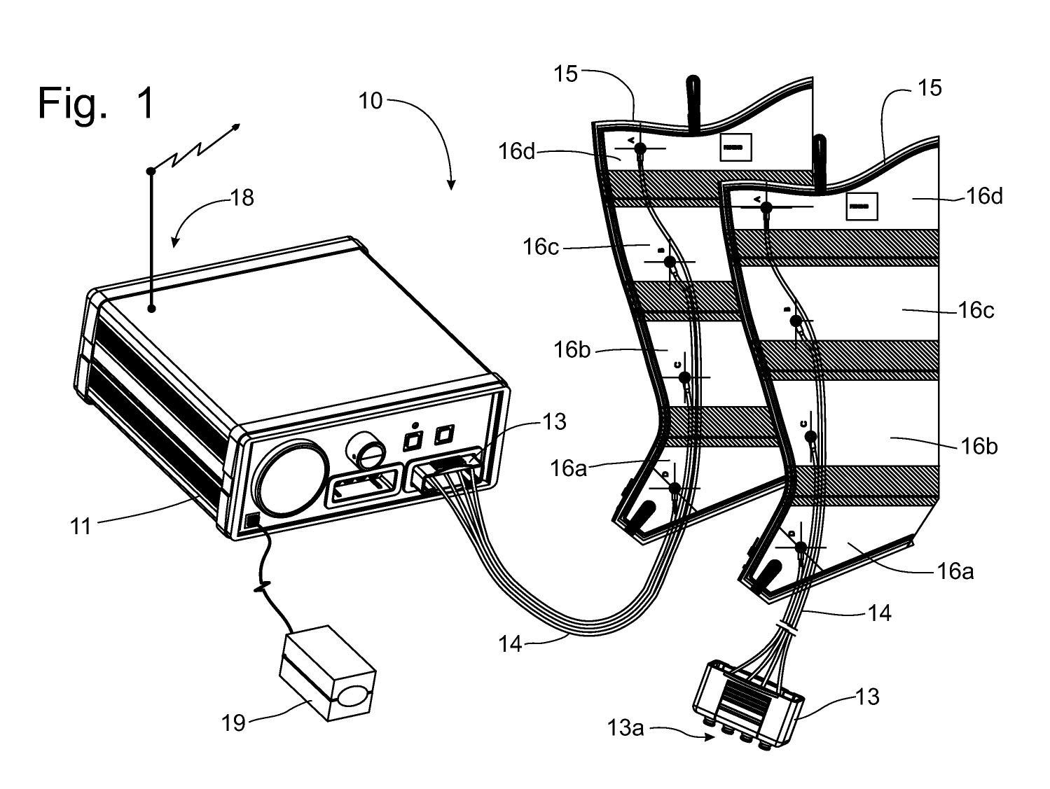

Referring to the FIGS. 1-6, representative medical devices, incorporating the principles of the instant invention, can best be seen. The particular therapy devices shown in the drawings are representative of medical devices that are typically used in home-based therapy, but not intended to be exclusive thereof. For example, the defibrillator 50 schematically shown in FIG. 6 is of the type that is deployed throughout airports and shopping malls to provide emergency service when needed. In FIG. 1, the medical apparatus 10 includes an electrically powered pump 11 coupled to inflatable garments 15 by respective conduit assemblies 12 having a clip 13 formed with multiple ports 13a connected to individual conduit lines 14. Each of the conduit lines 14 are coupled to a corresponding one of the inflation cells 16a-16d.

The pump 11 is programmed to divert air under pressure to the inflation cell 16a most distant from the patient's heart, which because the depicted garments 15 are intended for utilization on a patient's lower legs is the lowermost inflation cell 16a. The pump 11 then inflates the next higher inflation cell 16b until the entire garment 15 is inflated, whereupon all of the inflation cells 16a-16d are deflated and the cycle of inflation of the cells 16a-16d restarts. The sequential pressurization of the inflation cells 16a-16d aids the circulation of blood from the veins in the extremity back to the patient's heart. By coupling a pulse oximeter 19 to the pump 11, the pump can measure the percentage of oxygen saturation in the patient's blood and to sense the patient's heart beat. In addition, the pulse oximeter 19 provides positive feedback to the patient in the form of the oxygen saturation percentage to provide an indication that the therapy is working.

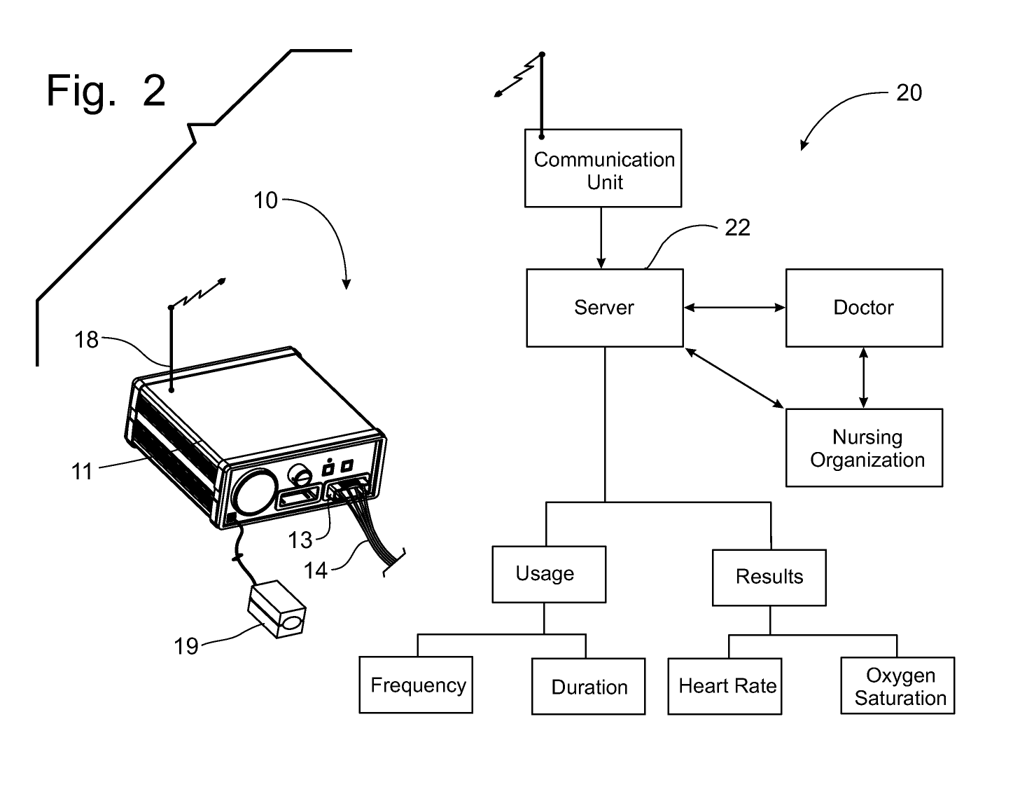

Referring now to FIG. 2, the pump 11 incorporates an integral communications device that communicates directly via a GSM network, or the equivalent thereof, to a Cloud server or directly to a professional medical caregiver without requiring the patient or other human interaction. Thus, whenever the medical device 10 is operating, or at least powered, the communications unit 12 can report the operational and informational data to the Cloud server 22 where the data can be stored in a coded manner and accessed by the professional health caregiver when desired. Accordingly, the monitoring system 20 will receive a signal directly from the communications device 12 that would provide an indication of utilization of the therapy device and the, such as when and for how long the medical device 10 was operated, as well as informational data such as heart rate and the oxygen saturation percentage of the patient.

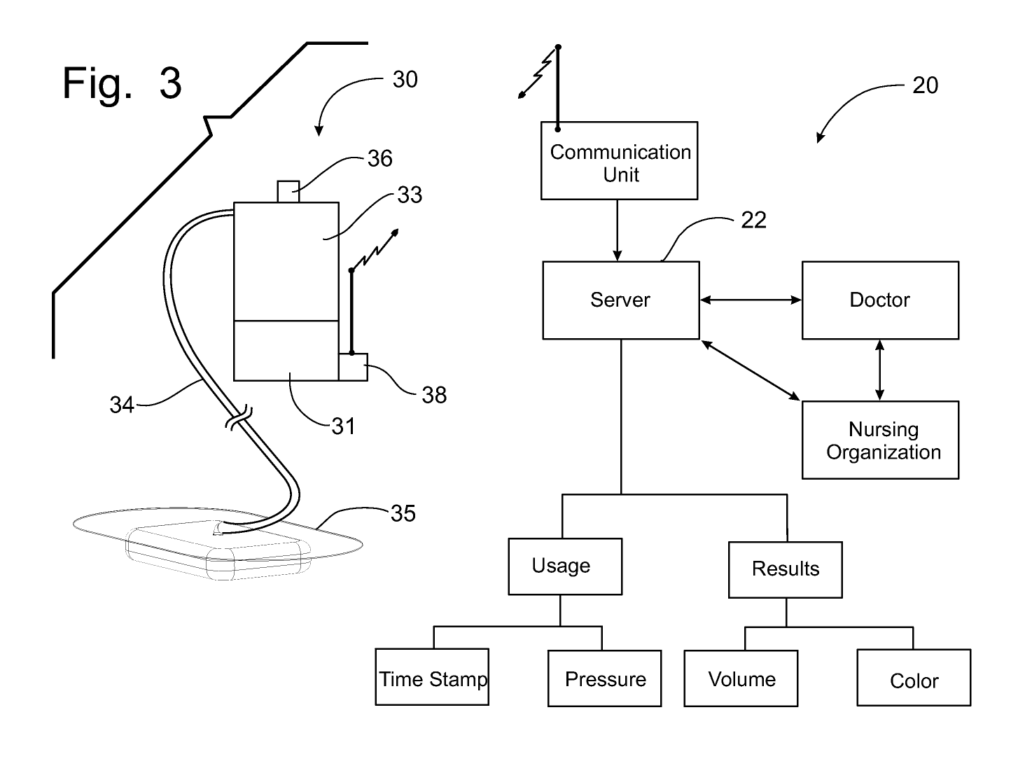

Other medical devices or home-based therapies can also be monitored in a similar manner. One such example is shown in FIG. 3, which is an in-home therapy consisting of a negative pressure bandage apparatus 30 that utilizes a pump 31 to draw a negative pressure on a bandage 35 placed on an open wound of the patient and sealed against the skin around the wound. The negative pressure draws the exudates from the wound into the bandage, or into a storage container 33 mounted on the pump 31, which are typically carried on the person of the patient remotely from the negative pressure bandage 35. Tubing 34 interconnecting the bandage 35 and the storage container 33 conveys the exudates into the storage container 33. A sensor or sensors 36 associated with the storage container 33 can sense a quality of the exudates, for example the color of the exudates, as well as a volume of the exudates collected within the storage container 33.

The communications unit 38 associated with the negative pressure bandage apparatus 30 communicates with the Cloud computing and storage unit and monitoring system 20 to transfer on a periodic basis, or even on a continuous basis, data related to the operation and effectiveness of the therapy apparatus 30. In the way of example, the information received and stored in the Cloud server 22 could provide a time stamp for the particular data being transmitted which would include the sensed quality of the exudates and the volume of the exudates collected. The differential in volume over a measured period of time provides an ongoing collection rate of the exudates from the wound. If the collection rate is above a predetermined parameter a problem with the wound can be identified. Similarly, if the color of the exudates is red, as opposed to a non-red color, the exudates being collected is likely blood, giving an indication that the wound is bleeding more than expected. A collection rate that is too low can indicate that the negative pressure bandage apparatus is not working properly.

Another parameter that can be transmitted to the monitoring system 20 is the pressure gradient being applied by the pump 31. If the pressure gradient is lower than necessary to make the negative pressure bandage 35 operable, there could be a problem with the seal of the bandage 35 against the patient's skin around the wound, or an indication that the pump is not working properly. In either case, a visit from the visiting nurse organization is likely called for to determine the problem.

Accordingly, the informational data received from a medical device could include ON/OFF flags, the duration where the therapy was on, intentional interruption of therapy, erratic therapy behavior, or error messages, and for a negative wound therapy device or an intermittent pneumatic compression therapy device, the informational data received can include ON/OFF flags for the device and prolonged irregular inactivity of the device, which informational data will be transmitted to the data cloud.

Since the operational and informational data is being stored on a Cloud server, the data can be run through predictive modeling software to provide analysis of the data and enhance the evaluation of the care being given and the care that needs to be given to the patient. The professional medical caregiver would have the ability to decode the data on the Cloud server to associate the data with a particular patient and thus would have access to the data and any reports generated by the predictive modeling software. In addition, the data can be filtered as the data is being received by the Cloud server through the predictive modeling software to raise an alarm directly with the professional medical caregiver when the data received is outside of a predetermined range of values. For example, if the operational data indicates that the pump cannot draw enough vacuum, the software can provide an alert to the professional caregiver, who can call the patient or make a visit to the patient's home to correct the leak. As another example, if the medical device 30 sends data to the Cloud server that indicates that a greater volume of fluids is being collected that would be expected, then the software will generate an alert and a professional caregiver can visit the patient to inspect the wound.

The professional caregiver might desire to view the raw data to obtain some insight as to the proper operation of the therapy 30. If the wound is healing properly, the volume of exudates collected in the storage container 33 would be expected to decrease over time. Such parameters can also be set within the server 22 to provide appropriate indicators to the health care providers. Also, negative pressure bandage apparatus 30 can be used to detect a problem with the wound. If a sudden and dramatic increase in the volume of the exudates collected is detected, a significant problem could be the cause, which can be obviated by an alert issued to the doctor and/or the visiting nurse organization by the server 22.

Many other in-home therapies can be appropriately monitored in a similar manner. Even the dispensing of medication from an appropriately designed medication dispenser having a communication unit (not shown) that would provide a signal to the monitoring system 20 that the medications have been dispensed at the appropriate time. Obviously, the monitoring system will not be able to ascertain that the patient has actually taken the prescribed medication, but the doctor and/or nurse will have feedback from the monitoring system 20 as to the dispensing of the medication. Other possible in-home therapy that could benefit from an application of the monitoring system could be an application of intravenous drugs which could be monitored with an apparatus that identifies the timing of the IV therapy and the volume of the drug administered through the IV set-up.

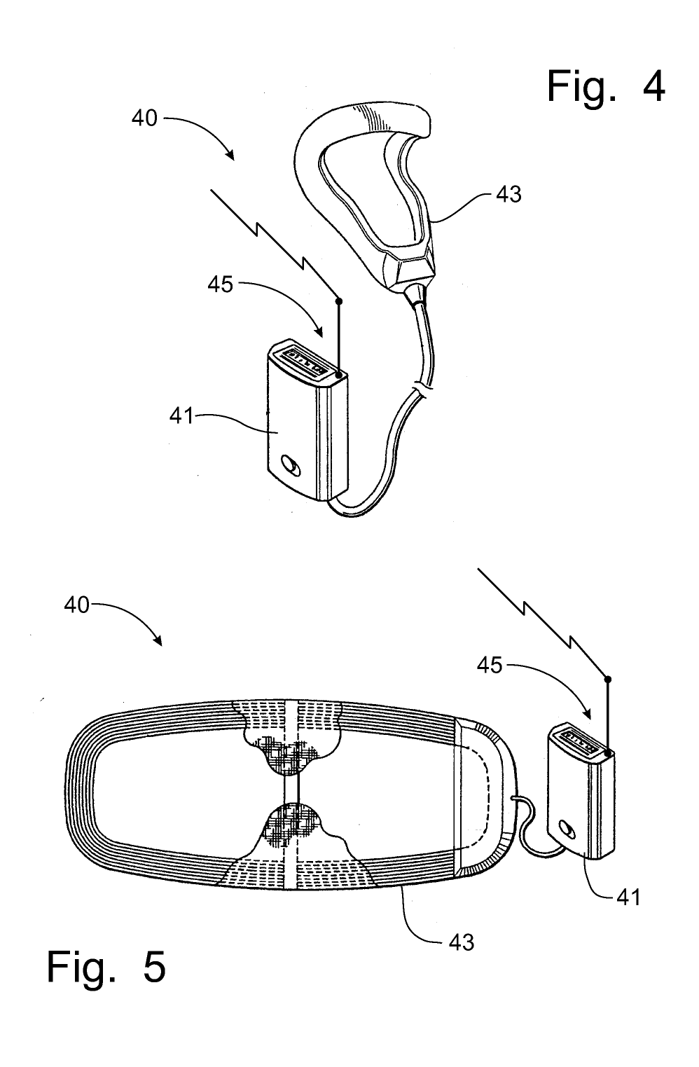

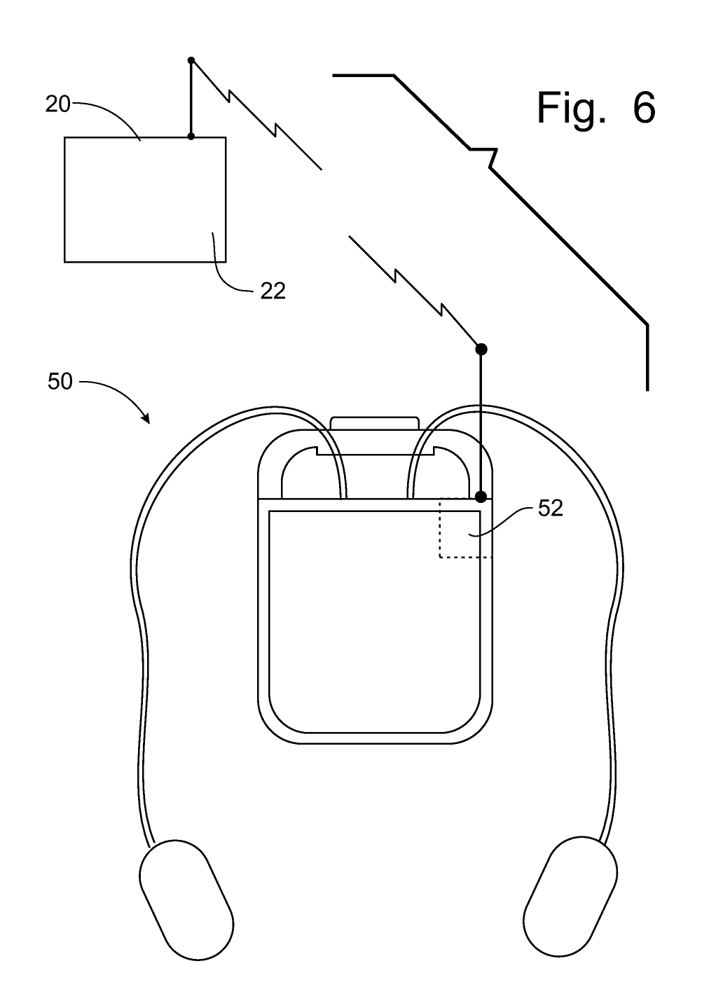

Bone growth stimulation therapy is typically applied in the home environment. Electromagnetic bone stimulators 40 are used to apply an electromagnetic field to the site for spinal fusion surgery to stimulate bone growth that fuses the vertebrae together. Such devices, as well as electrical stimulation devices, are typically battery powered and have a control module 41 that regulates the on/off function to send electrical current to a transducer 43 for the generation of an electromagnetic field. The control module 41 can determine the duration of operation of the therapy device 40. An application of a communications transmitter 45 to the control module 41, as is schematically represented in FIGS. 4 and 5, would permit the operation of the bone stimulation therapy device 40 to be monitored. Thus, the patient, as well as the medical professional responsible for the care of the patient, can monitor the operation of the therapy device 40 to determine if the device 40 is being used sufficiently often and for adequate durations to provide proper bone growth therapy.

Through a monitoring system 20 communicating to an off-site server 22 that stores the data transmitted to the monitoring system 20, the health care providers will have real data indicative of the utilization of the therapy, as well as the results obtained through the application of the therapy to the patient. With this data, a health care provider can more effectively determine the parameters by which the therapy is to be conducted. For example, in the inflatable garment therapy apparatus 10, the patient's doctor could decide to change the frequency or duration of the application of the therapy, or even change the timing relative to the sensing of the heart beat for the inflation of the inflation cells to provide a more effective operation of the inflatable garment 15.

The medical device 10 can communicate with a Cloud computing and storage site through a cellular network to store the data relating to the use and effectiveness of operation of the apparatus 10. The apparatus 10 can transmit to the Cloud computing and storage site when (and if) the apparatus is turned on and whether the apparatus 10 is functioning. Other information that can be transmitted to the Cloud computing and storage site are the operational parameters which are noted above. The Cloud computing and storage site can be accessed as needed by the appropriate medical professional who can review the transmitted data and determine if any action needs to be taken, or if a medical professional needs to visit the patient. The Cloud computing and storage site can incorporate an alert to notify the medical professional if any particular operational parameter is outside of an acceptable range. By incorporating a minor processing capability within the monitoring system 20 or within the medical device 10, the medical device 10 can send an alert to a predetermined location in the event an operational parameter is outside of a predetermined range.

Also, the use of a cellular GSM network to transmit data to a Cloud computing and storage site would permit the direct contact of the apparatus 10 by a medical professional to seek operational information from the apparatus 10, assuming that the apparatus 10 was powered. Under certain circumstances, the apparatus 10 can be configured to allow a medical professional to contact the medical device to query the device as to certain data or status of the device, as well as remotely change certain operative parameters of the apparatus 10 through communication thereto via the cellular network. For example, the operating pressure of a pump associated with the apparatus 10 can be modified if security provisions are appropriately resolved. The low power transmission of data through the cellular network provides a great deal of flexibility in the monitoring and control of a medical apparatus 10 being operated at home by a patient.

With this stored information, the doctor can retrieve the data and/or a report relating to the data to determine the utilization rate of the therapy, with regard to the length of the therapy as well as the frequency of the therapy, and the effectiveness of the therapy. The doctor can utilize that information to make a determination of whether to continue the therapy or to change the frequency or utilization of the therapy. When the doctor is consulting with the patient about the effectiveness of the therapy, the doctor will at least know that the patient is using the therapy and how often the therapy is being used. Utilization frequency alone is an important factor derived from the data stored on the server 22 as the doctor has no other means by which such information can be obtained.

Furthermore, if the server 22 receives a heart rate or oxygen saturation percentage parameter that is indicative of a possible problem with the patient, the server 22 can communicate with the doctor and possibly with the visiting nurse organization to provide an alert that a possible emergency situation exists. The doctor and/or the visiting nurse organization can then make contact with the patient to confirm if the therapy is being conducted properly, or to confirm that a problem exists for which additional and/or immediate help is needed. Also, if the therapy is not being utilized within predetermined frequency or duration parameters, a communication to the doctor and/or the visiting nurse organization can provide an alert that the therapy is not being properly conducted, whereupon a subsequent contact with the patient can reinforce the need to use the therapy properly and according to the prescription for the therapy.

Accordingly, the professional caregiver could also be passively notified by the data center of any of the informational data through a text message, a phone call, a pager message, a push mail on a smart phone, or a pop up notification on a computer screen or smart phone screen. As a result, the professional caregiver can provide intervention to the patient through various models through the cloud computing pathway, including the sending of a command back to the medical device using a smart phone or a computer to remotely control the medical device to start or stop, or to change settings, or to directly intervene with the patient by giving the patient a phone call upon the receipt of an error message by caregiver, or performing an onsite visit if situation warrants.

Referring now to the schematic representation of FIG. 6, a defibrillator 50 of the type deployed at public places, such as airports and shopping malls, for emergency usage is shown. The incorporation of a communications device 52 into the defibrillator 50 will enable the communications device 52 to report to a monitoring station 20 or to a Cloud server 22 the operational status of the defibrillator 50. For example, the defibrillator 50 can report through the communications device 52 that the software is operational and that the battery is charged adequately. Alternatively, the communications device 52 can be queried from the central monitoring station 20 to report on the operational status of the defibrillator 50. In response to that report, whether sent on a regular basis or as a result of a direct inquiry, the people responsible for the service and maintenance of the defibrillator 50 can make a determination as to whether the defibrillator 50 should be serviced in some manner to maintain the defibrillator 50 in an operative condition for a predetermined period of time.

In addition, the communications device 52 can be utilized to connect the operator of the defibrillator 50 with a professional caregiver to provide instructions as to how to administer treatment with the defibrillator 50. Also, the defibrillator 50 can be used as a sensor to ascertain the patient's heart rate and rhythm and perhaps other pertinent health data to provide assistance as to whether treatment with the defibrillator should be administered or not. The communications device 52 can also alert the central monitoring station 20 that the defibrillator 50 has been placed into use and, therefore, needs to be serviced as appropriate.

The use of iphones or smart phones to bridge the communication between the medical device and the professional caregiver is not particularly effective because the use of these devices requires input or activity from the patient. Furthermore, standalone medical device deployed for emergency service, such as defibrillators, do not have a human associated with the device to place the call to the central monitoring station or Cloud server. Thus, it is important that the communications device be capable of communicating directly with the Cloud server at regular intervals or upon usage of the device, etc. It is also advantageous that the communications device be capable of receiving an inquiry from the professional caregiver. Accordingly, each respective medical device incorporating the principles of the instant invention must have a unique identifier associated with the medical device and the communication device associated therewith.

Therefore, the process of communication between the medical device and the Cloud server is self-contained so that every medical device can communicate directly with the server and no patient or other human interface or cooperation is necessary to make the communication work. The principle of the instant invention can be applied to substantially any medical device that is powered either through electrical current, such as 120 volt house current, or through a battery, including blood pressure cuffs, temperature indicators, oxygen level indicators (oximeters) blood sugar testers, lymph edema pumps, negative pressure wound therapy systems, and defibrillators, as representative examples.

The principles of the instant invention would be effective in a hospital environment wherein many data collection devices, such as blood pressure cuffs, temperature indicators and oxygen level indicators, intravenous devices, etc., are utilized for patients, particularly following surgery. A communications device placed on each respective medical device can communicate the collected data to a central station and stored in a server, or even into a Cloud server. The data collected can be filtered and alerts issued in the same manner described above with respect to home-based therapy. Errors in transcriptions of the data from one clipboard to another would be avoided. Analytical reports from appropriate software analyzing the collected data can be made available. By incorporating some processing capability into the communications device or into the medical device, the medical device can issue an alert to an appropriate predetermined location when a medical parameter is outside of a predetermined range for that medical parameter.

The invention of this application has been described above both generically and with regard to specific embodiments. Although the invention has been set forth in what is believed to be the preferred embodiments, a wide variety of alternatives known to those of skill in the art can be selected within the generic disclosure.

Having thus described the invention, what is claimed is:

1. A powered medical device having an operative function that is expressed in terms of operational data, comprising:
a communications transmitter operatively connected to said powered medical device, said communications transmitter being operable to transmit said operational data directly to a remote station utilizing a communications network.

2. The powered medical device of claim 1 wherein said communications transmitter has a unique identifier.

3. The powered medical device of claim 2 wherein said powered medical device is a portable defibrillator, said operational data being selected from a group consisting of status of battery power, operational status of said portable defibrillator, and status of software associated with the operation of said portable defibrillator.

4. The powered medical device of claim 3 wherein said communications transmitter sends said operational data to said remote station on a predetermined schedule.

5. The powered medical device of claim 3 wherein said communications transmitter sends an alert to said remote station when the battery level reaches a predetermined parameter.

6. The powered medical device of claim 3 wherein said communications transmitter sends an alert to said remote station when said operational data of said portable defibrillator is indicative of said portable defibrillator being inoperative.

7. The powered medical device of claim 2 wherein said medical device is engaged with a human patient, said operational data being medical data of the human patient, said communications transmitter sending said medical data to said remote station to be recorded.

8. The powered medical device of claim 7 wherein said medical device is operated in a hospital setting, said medical data being data relating to the human patient and is selected from a group consisting of blood pressure, heart rate, heart rhythms, blood oxygen saturation, temperature, volume of fluids injected intravenously, and volume of bodily fluid removed from body.

9. The powered medical device of claim 8 wherein said remote station is a computer server accessible by hospital professional medical caregivers.

10. The powered medical device of claim 9 wherein said computer server sends an alert when any said medical data falls outside a predetermined range.

11. The powered medical device of claim 9 wherein said computer server is a Cloud server located remotely from the hospital setting.

12. The powered medical device of claim 2 wherein said medical device provides home-based medical therapy to a human patient, said operational data being medical data of the human patient, said remote station being a remote computer server receiving direct communications from said communications transmitter to record said medical data.

13. The powered medical device of claim 12 wherein said powered medical device is a negative pressure wound therapy system including a pump operable to exert a vacuum on a remote bandage applied to the human patient to collect said exudates from a wound on said human patient, said medical data being selected from a group consisting of pressure applied by said pump, volume of flow of said exudates from said wound, color of said exudates, and the duration of use of the pump.

14. The powered medical device of claim 12 wherein said powered medical device is an inflatable garment of the intermittent pneumatic compression therapy system, including a pump and a controller for applying pressurized air to an inflatable garment worn by the patient, said medical data relating to the operation of the inflatable garment therapy system, said medical data being selected from a group consisting of the heart rate of the patient, the length of time the therapy is utilized and the frequency of operation of the therapy.

15. The powered medical device of claim 14 wherein said powered medical device is an electromagnetic bone growth stimulator including a controller controlling the application of electricity to a transducer, said medical data being selected from a group consisting of frequency and duration of use of the electromagnetic bone growth stimulator.

16. The powered medical device of claim 2 wherein the communications network is a cellular network or WIFI network operable to transmit said operational data directly to a Cloud computing and storage site for access by a medical professional.

17. A method of monitoring the operation of a powered medical device having an operative function that is expressed in terms of operational data, comprising the steps of:
sensing said operational data of said powered medical device; and
transmitting said operational data from a communications device operatively coupled to said powered medical device to communicate said operational data directly to a remote Cloud computing and storage site, said communications device having a unique identifier to distinguish each said communications device from all other communications devices.

18. The method of claim 17 wherein operational data is compared to a predetermined range for said operational data to determine the operative effectiveness of said powered medical device.

19. The method of claim 18 wherein said operational data is medical data associated with a human patient utilizing said powered medical device, said medical data being analyzed to establish a diagnosis for said human patient.

20. The method of claim 18 wherein said powered medical device is a portable defibrillator, said operational data being transmitted to said Cloud computing and storage site on a predetermined schedule, said operational data being selected from a group consisting of status of battery power, operational status of said portable defibrillator, and status of software associated with the operation of said portable defibrillator.

21. The method of claim 18 wherein said powered medical device is engaged with a human patient, said operational data being medical data relating to the human patient and is selected from a group consisting of blood pressure, heart rate, heart rhythms, blood oxygen saturation, temperature, volume of fluids injected intravenously, and the volume of bodily fluid removed from body.

22. The method of claim 18 wherein said medical device provides home-based medical therapy to a human patient, said operational data being medical data of the human patient relating to said medical therapy, said medical device being one of a group consisting of a negative pressure wound therapy system including a pump operable to exert a vacuum on a remote bandage applied to the human patient to collect said exudates from a wound on said human patient, an inflatable garment therapy system including a pump and a controller for applying pressurized air to an inflatable garment worn by the patient, and an electromagnetic bone growth stimulator including a controller controlling the application of electricity to a transducer.

23. The method of claim 17 further comprising the step of:
accessing said operational data by a professional caregiver via a remote communications device selected from a group consisting of a smart phone application, a computer program, a website, and an online portal of a hospital intranet.

24. The method of claim 23 wherein said accessing step includes the step of passively notifying said professional caregiver by the Cloud computing and storage site of operational data collected from the monitored medical device by communications via one of a group consisting of a text message, a phone call, a pager message, a push mail on a smart phone, or a pop up notification on a computer screen or smart phone screen.

25. The method of claim 23 further comprising the step of:
intervening with a human patient utilizing said powered medical device by sending commands to said powered medical device to change the operative function of said powered medical device.

26. The method of claim 25 wherein said intervening step is conducted through the Cloud computing and storage site by remotely transmitting operational command signals through the Cloud computing and storage site to control the operative functions of said powered medical device.

27. The method of claim 26 wherein the operational command signals sent to said powered medical device are selected from a group consisting of controlling an ON/OFF function of said powered medical device and adjusting operational settings.
